(19) United States
(12) Patent Application Publication
(10) Pub. No.: US 2026/0151048 A1
(43) Pub. Date: Jun. 4, 2026

(54) BREATH SENSOR, BREATH SENSING SYSTEM, BREATH SENSING METHOD, AND NON-TRANSITORY COMPUTER-READABLE MEDIUM

(71) Applicant: Asahi Kasei Microdevices Corporation, Tokyo (JP)

(72) Inventors: Naoya UGOMORI, Tokyo (JP); Yuta TAKAGI, Tokyo (JP)

(21) Appl. No.: 19/394,884

(22) Filed: Nov. 20, 2025

(30) Foreign Application Priority Data
Nov. 29, 2024 (JP) 2024-208993
Oct. 21, 2025 (JP) 2025-176872

(51) Int. Cl. A61B 5/087; A61B 5/00

(52) U.S. Cl. CPC A61B 5/0873; A61B 5/0082; A61B 5/7264; A61B 5/7271; A61B 5/7405; A61B 5/742; A61B 5/7455

(57) ABSTRACT

There is provided a breath sensing system including: a breath sensor which senses exhaled air, the breath sensor having a light emission unit which emits light that is directed toward a path through which the exhaled air passes, and a light receiving unit which receives at least a part of the light, and which outputs a light receiving signal; a respiration information calculation unit which calculates respiration information; a reliability calculation unit which calculates reliability level information; an output unit which outputs the respiration information and the reliability level information; a receiving unit which receives the respiration information and the reliability level information; a movable unit which is provided with the breath sensor, and which has a variable position; and a notification unit which provides a notification of information related to the movable unit, according to the reliability level information.

The contents of the following patent application(s) are incorporated herein by reference:
NO. 2024-208993 filed in JP on Nov. 29, 2024.
NO. 2025-176872 filed in JP on Oct. 21, 2025.

### BACKGROUND

### 1. Technical Field

The present invention relates to a breath sensor, a breath sensing system, a breath sensing method, and a non-transitory computer-readable medium.

### 2. Related Art

Patent Document 1 discloses a “breath test system capable of measuring a concentration of carbon dioxide contained in exhaled air”.

### RELATED ART DOCUMENTS

### Patent Document

Patent Document 1: Japanese Patent Application Publication No. 2017-187365
Patent Document 2: Japanese Patent No. 5351583
Patent Document 3: Japanese Patent Application Publication No. 2022-003321
Patent Document 4: Japanese Patent No. 3627243
Patent Document 5: Japanese Patent Application Publication No. 2024-010292

### BRIEF DESCRIPTION OF THE DRAWINGS

FIG. 1 shows an overview of a configuration of a breath sensor 100.

FIG. 2A shows an example of signal processing.

FIG. 2B shows an example of a determination signal that is calculated from a light receiving signal ratio and a baseline shown in FIG. 2A.

FIG. 3A shows an example of the breath sensor 100.

FIG. 3B shows a modified example of the breath sensor 100.

FIG. 4 shows an example of a light emission unit 110.

FIG. 5 shows an overview of a configuration of a breath sensing system 10.

FIG. 6A shows an example of an operation of the breath sensing system 10.

FIG. 6B shows an example of an operation of the breath sensing system 10.

FIG. 6C shows an example of an operation of the breath sensing system 10.

FIG. 7A shows an example of a usage state of a headset 500 including the breath sensor 100.

FIG. 7B shows an example of a usage state of the headset 500 including the breath sensor 100.

FIG. 7C shows an example of a usage state of the headset 500 including the breath sensor 100.

FIG. 8 shows an example of a usage state of a head-mounted display 600 including the breath sensor 100.

FIG. 9A shows an example of a clip-type apparatus 800 including the breath sensor 100.

FIG. 9B shows an example of a usage state of the clip-type apparatus 800 including the breath sensor 100.

FIG. 9C shows an example of a usage state of the clip-type apparatus 800 including the breath sensor 100.

FIG. 10 shows an overview of a configuration of the breath sensor 100 and a calculation apparatus 700.

FIG. 11 shows an example of a computer 1000 in which a plurality of aspects of the present invention may be embodied entirely or partially.

### DESCRIPTION OF EXEMPLARY EMBODIMENTS

Hereinafter, the present invention will be described through embodiments of the invention, but the following embodiments do not limit the invention according to the claims. In addition, not all combinations of features described in the embodiments are essential to a solution of the invention.

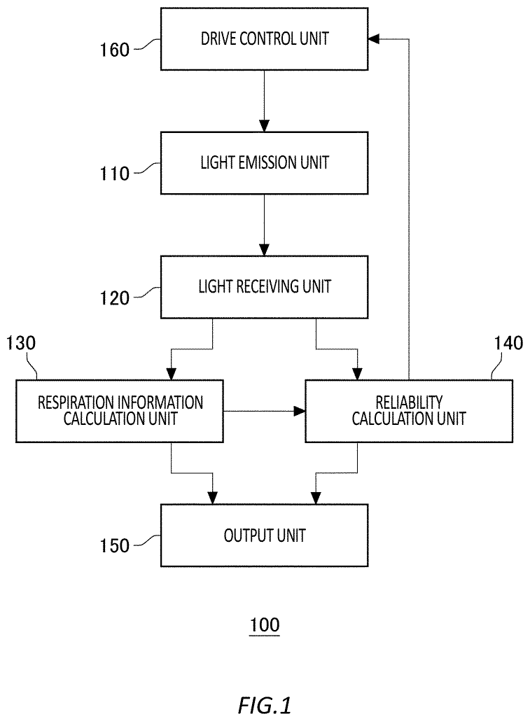

FIG. 1 shows an overview of a configuration of a breath sensor 100. The breath sensor 100 includes a light emission unit 110, a light receiving unit 120, a respiration information calculation unit 130, a reliability calculation unit 140, and an output unit 150. The breath sensor 100 may include a drive control unit 160. It should be noted that the illustrated blocks are functional blocks that are functionally separated from each other, and may not necessarily match actual apparatus configurations. That is, in the present figure, a block illustrated as one block may not necessarily be configured by one apparatus. In addition, blocks illustrated as separate blocks in the present figure may not necessarily be configured by separate apparatuses. The same applies to blocks in other figures.

The breath sensor 100 senses exhaled air generated by respiration. The breath sensor 100 may sense exhaled air that is generated by respiration of a living body, such as a human or a pet including a dog or a cat. As an example, the breath sensor 100 is an NDIR (non-dispersive infrared) sensor which utilizes a characteristic absorption wavelength of carbon dioxide contained in the exhaled air. However, a type of the breath sensor 100 is not limited to this.

The light emission unit 110 emits light that is directed toward a path through which the exhaled air passes. As an example, the light emission unit 110 includes an IR-LED. The light that is emitted by the light emission unit 110 may be infrared light, or may be light including at least the absorption wavelength of carbon dioxide. A part of the light emitted by the light emission unit 110 may pass through the path through which the exhaled air passes, and another part of the light emitted by the light emission unit 110 may not pass through the path through which the exhaled air passes. That is, a part of the light emitted by the light emission unit 110 may be directed toward the path through which the exhaled air passes, and another part of the light emitted by the light emission unit 110 may not be directed toward the path through which the exhaled air passes. The light emitted by the light emission unit 110 may be directly directed toward the path through which the exhaled air passes, or may be directed via a light guide unit such as a mirror toward the path through which the exhaled air passes.

The light receiving unit 120 receives at least a part of the light emitted by the light emission unit 110, and outputs a light receiving signal corresponding to a light receiving result. As an example, the light receiving unit 120 includes a photodiode. The light receiving unit 120 may receive, among light emitted by the light emission unit 110, light that has passed through the path through which the exhaled air passes. The light receiving unit 120 may receive, among light emitted by the light emission unit 110, both of the light that has passed through the path through which the exhaled air passes and light that has not passed through the path through which the exhaled air passes. The light receiving unit 120 may supply the light receiving signal corresponding to the light receiving result to the respiration information calculation unit 130 and the reliability calculation unit 140.

The light receiving unit 120 may supply the light receiving signal processed according to the light receiving result to the respiration information calculation unit 130 and the reliability calculation unit 140. For example, the light receiving unit 120 may include a processing circuit, and may supply a signal processed by the processing circuit to the respiration information calculation unit 130 and the reliability calculation unit 140. As another example, the breath sensor 100 may include a signal processing unit which processes the light receiving signal; and the light receiving unit 120 may supply, via the signal processing unit, the light receiving signal corresponding to the light receiving result to the respiration information calculation unit 130 and the reliability calculation unit 140. As yet another example, the respiration information calculation unit 130 and the reliability calculation unit 140 to which the light receiving signal corresponding to the light receiving result is supplied, may perform signal processing respectively.

As described above, a configuration for processing the light receiving signal is not particularly limited. However, the respiration information calculation unit 130 and the reliability calculation unit 140 may use a light receiving signal that is not processed.

The respiration information calculation unit 130 calculates respiration information related to the respiration, based on the light receiving signal. The respiration information calculation unit 130 may calculate at least one of a period or a duration of the respiration. The respiration information calculation unit 130 may calculate the respiration information including a duration time of exhalation, a duration time of inhalation, or a depth of the respiration. An amount of light received is decreased at the absorption wavelength of carbon dioxide by carbon dioxide absorbing the light. The respiration information calculation unit 130 may calculate the respiration information related to the respiration, based on a change in intensity of the light receiving signal due to the absorption of light by carbon dioxide. The respiration information calculation unit 130 may supply the calculated respiration information to the reliability calculation unit 140 and the output unit 150.

The reliability calculation unit 140 calculates reliability level information indicating reliability of the respiration information, based on the light receiving signal. For example, the reliability calculation unit 140 may calculate the reliability level information based on a signal or a signal-to-noise ratio of the light receiving signal. The reliability calculation unit 140 may calculate the reliability level information based on the respiration information. The reliability calculation unit 140 may supply the calculated the reliability level information to the output unit 150.

The light receiving signal may include stationary noise. The noise may be environmental noise due to a temperature, wind, or the like in an environment where the breath sensor 100 is placed, or may be noise due to a drive of the light emission unit 110 or a measurement by the light receiving unit 120. The reliability of exhalation information may become high when signal intensity relative to noise intensity is great, and the reliability of the exhalation information may become low when the signal intensity relative to the noise intensity is small.

The reliability calculation unit 140 may calculate the reliability level information in two or more levels of abstraction. For example, the reliability calculation unit 140, as the reliability level information, calculates, when the reliability of the respiration information falls within a predetermined first range, that the reliability is at a first level. The reliability calculation unit 140, as the reliability level information, may calculate, when the reliability of the respiration information falls within a second range that has reliability higher than the first range, that the reliability is at a second level. The reliability calculation unit 140 may calculate the reliability level information in two levels of abstraction, may calculate the reliability level information in three levels of abstraction, or may calculate the reliability level information in four levels of abstraction. The number of levels of abstraction of the reliability level information that is calculated by the reliability calculation unit 140, is not particularly limited.

For example, the reliability calculation unit 140, as the reliability level information, calculates, in a case where the reliability calculation unit 140 calculates the reliability level information in two levels of abstraction, when the signal-to-noise ratio of the light receiving signal is lower than a predetermined reference value, that the reliability is at the first level. The reliability calculation unit 140, as the reliability level information, may calculate, when the signal-to-noise ratio of the light receiving signal is higher than or equal to the reference value, that the reliability is at the second level.

For example, the reliability calculation unit 140, as the reliability level information, calculates, in a case where the reliability calculation unit 140 calculates the reliability level information in three levels of abstraction, when the signal-to-noise ratio of the light receiving signal is lower than a predetermined first reference value, that the reliability is at the first level. The reliability calculation unit 140, as the reliability level information, may calculate, when the signal-to-noise ratio of the light receiving signal is higher than or equal to the first reference value, and is below a predetermined second reference value, that the reliability is at the second level. The reliability calculation unit 140, as the reliability level information, may calculate, when the signal-to-noise ratio of the light receiving signal is higher than or equal to the second reference value, that the reliability is at a third level. The number of levels of abstraction is similar in another case as well.

The reliability calculation unit 140 may calculate the reliability level information based on at least one of a period or a duration of the respiration included in the respiration information. For example, the reliability calculation unit 140 may calculate the reliability level information by comparing a variance of the period or the duration of the respiration obtained from a plurality of measurements with a predetermined reference value. For example, the reliability calculation unit 140, as the reliability level information, may calculate, when the variance of the respiration information is great and fluctuation is great, that the reliability of the respiration information is low.

The output unit 150 outputs the respiration information and the reliability level information. The output unit 150 may output the respiration information and the reliability level information to another element which constitutes a breath sensing system 10 which will be described below. The output unit 150 may output the respiration information and the reliability level information to a headset on which the breath sensor 100 is mounted, may output the respiration information and the reliability level information to a head-mounted display on which the breath sensor 100 is mounted, or may output the respiration information and the reliability level information to a clip-type apparatus on which the breath sensor 100 is mounted. The output unit 150 may be provided in the headset on which the breath sensor 100 is mounted, on the head-mounted display on which the breath sensor 100 is mounted, or on the clip-type apparatus on which the breath sensor 100 is mounted. In this case, the output unit 150 may output the respiration information and the reliability level information to an external computer connected, in a wired and/or wireless manner, to each apparatus on which the breath sensor 100 is mounted. The headset, the head-mounted display, and the clip-type apparatus may constitute at least a part of the breath sensing system 10.

The output unit 150 is an example of a transmission unit which transmits the information in a wired manner. The output unit 150 is an example of the transmission unit which transmits the information wirelessly. However, the breath sensor 100 may have the transmission unit separate from the output unit 150.

The output unit 150 may output the reliability level information at a time interval that is longer than a time interval at which the respiration information is output. For example, the output unit 150 outputs the respiration information at a time interval of several hundred milliseconds. The time interval at which the output unit 150 outputs the respiration information may be periodic, or may be non-periodic. The output unit 150 may output the reliability level information at a time interval of several seconds. The time interval at which the output unit 150 outputs the reliability level information may be periodic, or may be non-periodic.

The output unit 150 may include a plurality of output terminals. The output unit 150 may output the respiration information and the reliability level information from different output terminals. For example, the output unit 150 outputs the reliability level information from a first output terminal and outputs the respiration information from a second output terminal which is different from the first output terminal.

When the breath sensor is mounted on an existing system or apparatus to constitute the breath sensing system, the existing system or apparatus may need to determine the reliability of exhalation information in order to place the breath sensor at a position suitable to sense the exhalation. That is, when the breath sensor is mounted on the existing system or apparatus, the existing system or apparatus may have to be restructured to determine the reliability of the exhalation information. Alternatively, when the breath sensor is simply mounted on the existing system or apparatus, the breath sensor may remain placed at a position where the reliability of the exhalation information is low without the reliability of the exhalation information being determined.

The breath sensor 100 in the present example includes the reliability calculation unit 140 which calculates the reliability level information and the output unit 150 which outputs the reliability level information. In this manner, even when the breath sensor 100 is mounted on an existing system or apparatus to constitute the breath sensing system 10, it is possible to easily facilitate the placement of the breath sensor 100 at a position suitable to sense the exhalation. An operation of the breath sensing system 10 to place the breath sensor 100 at the suitable position will be described below.

In addition, in the breath sensor 100 in the present example, the reliability calculation unit 140 calculates the reliability level information in two or more levels of abstraction, and the output unit 150 outputs the reliability level information. This makes it possible to convey the reliability level information with a smaller amount of information than a case where raw data is output. For example, even when the breath sensor 100 is mounted on an existing system or apparatus to constitute the breath sensing system 10, it is possible to reduce a need for information processing on an existing system or apparatus side, and thus it is possible for the breath sensor 100 to be easily mounted on the existing system or apparatus.

The drive control unit 160 may control a drive of the light emission unit 110 based on the reliability level information. For example, the reliability level information includes the signal-to-noise ratio of the light receiving signal. In this case, the drive control unit 160 may control an amount of drive current for driving the light emission unit 110 based on the signal-to-noise ratio. As an example, the drive control unit 160 causes the amount of drive current to be increased when the signal-to-noise ratio falls below a predetermined reference value.

For example, when the signal-to-noise ratio is lower than a predetermined reference value, the noise is too high and thus it is not possible to accurately calculate the respiration information, and the reliability of the respiration information becomes low. In this case, the drive control unit 160 causes the amount of drive current to be increased, thereby strengthen the signal intensity of the light receiving signal, and enhancing the signal-to-noise ratio. This makes it possible to increase the reliability of the respiration information. The drive control unit 160 may also raise a drive speed for driving the light emission unit 110.

As another example, the drive control unit 160 may cause the amount of drive current to be decreased when the signal-to-noise ratio exceeds a predetermined reference value. When the signal-to-noise ratio exceeds the predetermined reference value, the reliability of the respiration information is high. In this case, the drive control unit 160 causes the amount of drive current to be decreased, thereby making it possible to reduce power consumption. The drive control unit 160 may also lower the drive speed for driving the light emission unit 110.

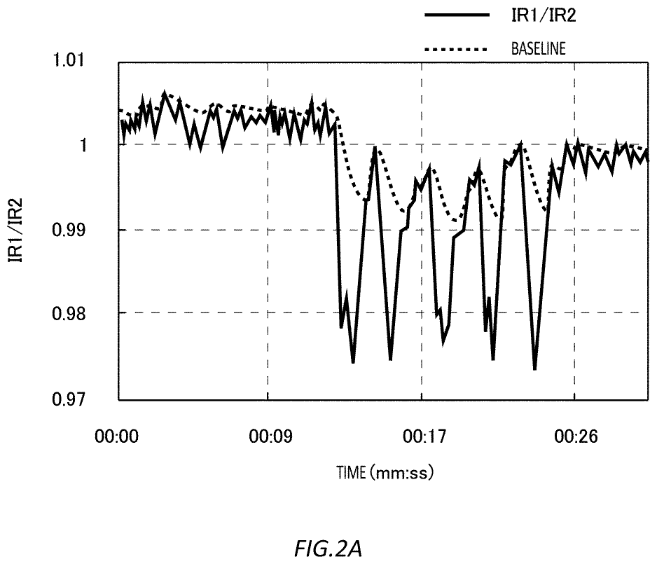

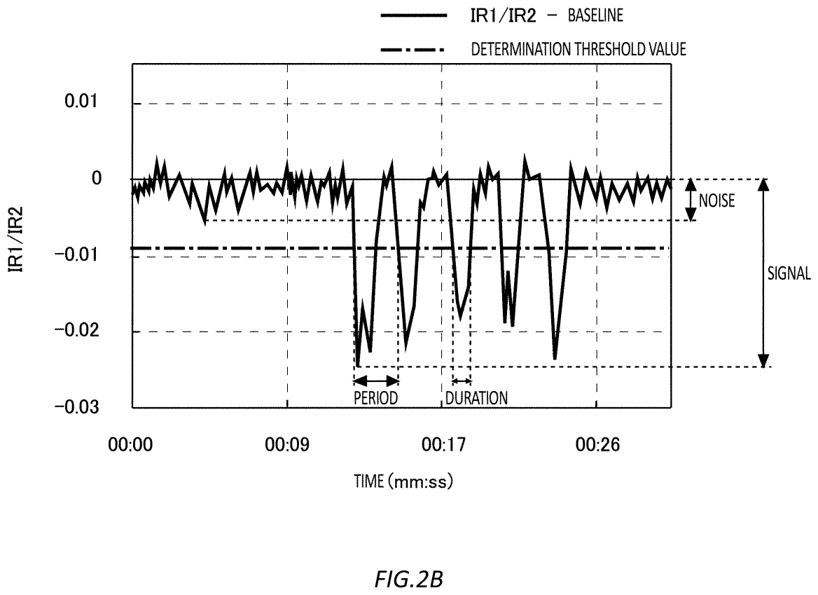

FIG. 2A shows an example of signal processing. As described above, the signal processing may be performed by the processing circuit included in the light receiving unit 120, may be performed by the signal processing unit included in the breath sensor 100, or may be performed by each of the respiration information calculation unit 130 and the reliability calculation unit 140.

In the present specification, a signal of the light which has passed through the path through which the exhaled air passes, among light emitted by the light emission unit 110, and which is received by the light receiving unit 120, is referred to as a first light receiving signal IR1; and a signal of the light which has not passed through the path through which the exhaled air passes, among light emitted by the light emission unit 110, and which is received by the light receiving unit 120, is referred to as a second light receiving signal IR2. A horizontal axis of FIG. 2A represents time, and a vertical axis represents a value of a ratio between the intensity of the first light receiving signal and the intensity of the second light receiving signal. In the present specification, the ratio between the intensity of the first light receiving signal and the intensity of the second light receiving signal, is referred to as a light receiving signal ratio. In addition, the intensity of the first light receiving signal may be simply referred to as the first light receiving signal and the intensity of the second light receiving signal may be simply referred to as the second light receiving signal. By taking the ratio between the first light receiving signal and the second light receiving signal, it is possible to correct an output fluctuation due to the change in the amount of light of the light emission unit 110 or due to the temperature.

The present example describes a case of using the light receiving signal ratio between the first light receiving signal and the second light receiving signal; however, the second light receiving signal may not be used. Instead of the light receiving signal ratio, a signal value of the first light receiving signal may be used. In this case, the term “light receiving signal ratio” may be used interchangeably with the “first light receiving signal”. That is, when the first light receiving signal is used instead of the light receiving signal ratio, processing similar to the processing described as processing, which is performed on the light receiving signal ratio in the present specification, may be performed on the first light receiving signal.

The signal processing for the light receiving signal ratio may include noise removal by a low-pass filter, and may include a baseline calculation. The baseline is a signal waveform due to a factor (disturbance) other than carbon dioxide contained in the exhaled air. The baseline is a waveform obtained by reducing or excluding a component due to a concentration of carbon dioxide from a waveform that is indicated by a solid line in FIG. 2A. The baseline may include a component of the intensity of the light that is emitted by the light emission unit 110, and a fluctuation component due to a factor other than a concentration change in carbon dioxide. When the breath sensor 100 senses the exhalation, the temperatures of the light emission unit 110 and the light receiving unit 120 may be changed by the temperature of the exhaled air, to change characteristics of the light emission unit 110 and the light receiving unit 120. The baseline may be a signal waveform that includes an influence of such a characteristic change and that does not include an influence of carbon dioxide. The baseline due to the disturbance outside a frequency of a respiration period may also include an influence such as humidity or degradation of an element.

For example, the baseline is calculated by filtering that is expressed by an Infinite Impulse Response (IIR). As another example, the baseline may be calculated by a moving average filter (MA: Moving Average), may be calculated by a weighted moving average filter (WMA: Weighted Moving Average), may be calculated by a finite impulse response filter (FIR: Finite Impulse Response), or may be calculated by a Butterworth filter. When a signal fluctuation is small, for example, the baseline may be calculated based on the signal value of data during a period when no respiration occurs.

In FIG. 2A, the solid line represents the light receiving signal ratio, and a dotted line represents the baseline. An amount of light received by the light receiving unit 120 is decreased by the absorption of carbon dioxide, and thus when the breath sensor 100 senses the exhalation, the light receiving signal ratio is decreased. The exhaled air quickly diffuses into the air, and thus the light receiving signal ratio is increased (recovered) immediately after the decrease. In FIG. 2A, the fluctuation in the light receiving signal ratio between 12 seconds and 25 seconds correspond to the respiration. One valley shape part in the light receiving signal ratio corresponds to one respiration. In the example of FIG. 2A, five valley shape parts appear in the waveform of the light receiving signal ratio, and thus indicate the light receiving signal ratios for five respirations.

FIG. 2B shows an example of a determination signal that is calculated from a light receiving signal ratio and a baseline shown in FIG. 2A. The signal processing for the light receiving signal ratio may include signal processing of removing the baseline from the light receiving signal ratio. Removing the baseline may mean subtracting, from the signal value at each point in time, the value of the baseline at that point in time. By removing the baseline, it is possible to correct the change in the signal value due to a factor other than carbon dioxide that is contained in the exhaled air, and it is possible to perform a more accurate measurement. In the present specification, a signal after the baseline is removed from the light receiving signal ratio may be referred to as a determination signal.

The determination signal indicates a signal component due to the exhalation, and thus the determination signal when no exhalation is present ideally coincides with a reference point (IR1/IR2=0). In addition, the determination signal when the exhalation is present ideally becomes smaller than the reference point.

In FIG. 2B, a solid line indicates the determination signal obtained by removing the baseline from the light receiving signal ratio, and a dash-single dotted line indicates a predetermined determination threshold value. As an example, when the signal value is lower than the predetermined determination threshold value, the breath sensor 100 determines that the exhalation is sensed.

The respiration information calculation unit 130 calculates the respiration information related to the respiration, based on the light receiving signal. The respiration information calculation unit 130 may calculates the respiration information related to the respiration, based on the determination signal. The respiration information calculation unit 130 may calculate at least one of a period or a duration of the respiration. For example, the respiration information calculation unit 130 calculates at least one of a period or a duration of the respiration, based on the determination signal.

The respiration information calculation unit 130 may set, as the duration of the respiration, one continuous period during which the value of the determination signal is lower than the determination threshold value. The respiration information calculation unit 130 may calculate, as the period of the respiration, a sum of one continuous period during which the value of the determination signal is higher than the determination threshold value, and one continuous period during which the value of the determination signal is lower than the determination threshold value. As the period or the duration of the respiration, the respiration information calculation unit 130 may calculate an average value in a predetermined period. By calculating the period or the duration of the respiration, it is possible to grasp psychological information regarding whether a human, or a pet or the like that is breathing is psychologically in an excited state or a relaxed state, or the like. when the period or the duration of the respiration is measured, the value of the determination signal and the determination threshold value only need to be able to be compared, and an amplitude of the determination signal may not be calculated, and a measurement precision of the value of the determination signal may not be high.

The reliability calculation unit 140 calculates the reliability level information indicating the reliability of the respiration information, based on the light receiving signal. The reliability calculation unit 140 may calculate the reliability level information indicating the reliability of the respiration information, based on the determination signal. The reliability calculation unit 140 may calculate the reliability level information based on the signal-to-noise ratio of the light receiving signal. The reliability calculation unit 140 may calculate the reliability level information based on the signal-to-noise ratio of the determination signal.

The signal intensity of the light receiving signal may be the signal intensity of the light receiving signal relative to the baseline. For example, the signal intensity of the light receiving signal is a maximum value of the signal intensity of the light receiving signal relative to the baseline. As another example, the signal intensity of the light receiving signal may be an average value of peak intensity of the light receiving signal relative to the baseline. The noise intensity of the light receiving signal may be the noise intensity relative to the baseline. For example, the noise intensity of the light receiving signal is a maximum value of the noise intensity relative to the baseline. As another example, the noise intensity of the light receiving signal may be an average value of noise peak intensity relative to the baseline. The signal-to-noise ratio of the light receiving signal may be a ratio of the signal intensity to the noise intensity. However, a method for calculating the signal-to-noise ratio is not particularly limited. It should be noted that the determination signal is obtained by subtracting the value of the baseline from the light receiving signal, and thus it is understood that the signal-to-noise ratio of the light receiving signal and the signal-to-noise ratio of the determination signal are substantially the same.

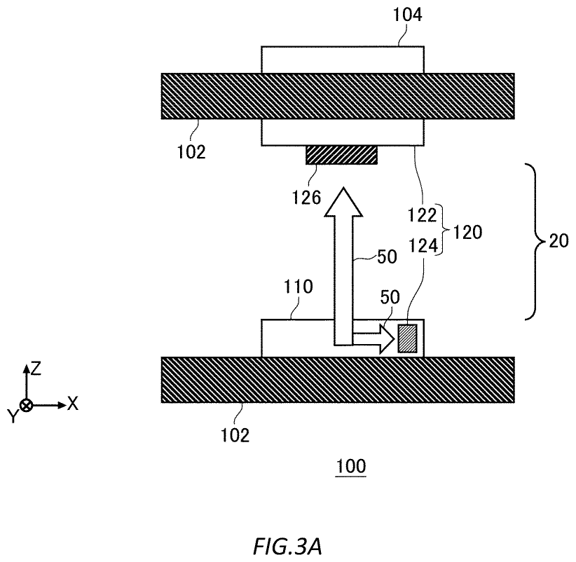

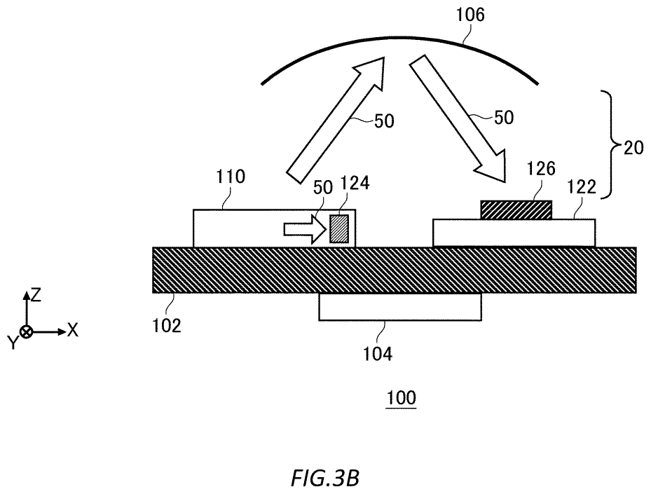

FIG. 3A shows an example of the breath sensor 100. The breath sensor 100 in the present example includes a support member 102 and a calculation unit 104. The light emission unit 110, the light receiving unit 120, and the calculation unit 104 may be mounted on the support member 102. In the present example, the light emission unit 110 is mounted on one of the support members 102 which face each other, and the calculation unit 104 is mounted on the other. In the present example, a plane parallel to a main surface of the support member 102 is set as an XY plane, and a direction perpendicular to the main surface of the support member 102 is set as a Z axis direction.

The light emission unit 110 emits light 50 that is directed toward a path 20 through which the exhaled air passes. The light receiving unit 120 receives at least a part of the light 50 emitted by the light emission unit 110, and outputs the light receiving signal corresponding to the light receiving result. The light receiving unit 120 in the present example may receive, among the light 50 emitted by the light emission unit 110, both of the light 50 that has passed through the path 20 through which the exhaled air passes and the light 50 that has not passed through the path 20 through which the exhaled air passes.

The light receiving unit 120 may have: a first light receiving element 122 mounted on the support member 102 on which the calculation unit 104 is mounted; and a second light receiving element 124 mounted on the support member 102 on which the light emission unit 110 is mounted. The first light receiving element 122 may receive, among the light 50 emitted by the light emission unit 110, the light 50 that has passed through the path 20 through which the exhaled air passes. That is, the first light receiving element 122 may output the first light receiving signal IR1. The second light receiving element 124 may receive, among the light 50 emitted by the light emission unit 110, the light 50 that has not passed through the path 20 through which the exhaled air passes. That is, the second light receiving element 124 may output the second light receiving signal IR2.

It should be noted that the light receiving unit 120 may not have the second light receiving element 124. For example, when instead of the light receiving signal ratio, the first light receiving signal is used, the light receiving unit 120 may not have the second light receiving element 124.

The breath sensor 100 may include an optical filter 126. The optical filter 126 may be arranged, on an optical path of the light 50, between the light emission unit 110 and the first light receiving element 122. In the present example, the optical filter 126 is provided on a surface of the first light receiving element 122. The optical filter 126 limits the wavelength of the light 50 that is incident on the first light receiving element 122. The optical filter 126 is a bandpass filter as an example.

Responsivity of the first light receiving element 122 may be changed under the influence of the temperature. In a responsivity curve with the horizontal axis representing the wavelength of the light and the vertical axis representing the responsivity of the first light receiving element 122, the influence of the temperature appears as a shift in the horizontal direction of the responsivity curve. The responsivity curve has a wavelength range in which a value of the responsivity is almost flat. By using the optical filter 126 to cause only light of a wavelength in that wavelength range to be incident on the first light receiving element 122, it is possible to suppress the influence on the responsivity even when the temperature of the first light receiving element 122 is changed.

The calculation unit 104 performs various calculations on the light receiving signal. The calculation unit 104 is an IC, as an example. The calculation unit 104 may calculate the light receiving signal ratio, may calculate the baseline, and may calculate the determination signal. The calculation unit 104 may function as the respiration information calculation unit 130 which calculates the respiration information related to the respiration, based on the light receiving signal. The calculation unit 104 may function as the reliability calculation unit 140 which calculates the reliability level information indicating the reliability of the respiration information, based on the light receiving signal. The calculation unit 104 may function as the output unit 150 which outputs the respiration information and the reliability level information.

FIG. 3B shows a modified example of the breath sensor 100. The breath sensor 100 in the present example differs from the example in FIG. 3A in that the light emission unit 110, the light receiving unit 120, and the calculation unit 104 are mounted on the common support member 102. In the present example, the differences from the example of FIG. 3A will be particularly described, and other configurations may be the same as those in the example of FIG. 3A.

The breath sensor 100 in the present example includes a mirror 106. A space between the support member 102 and the mirror 106 may be the path 20 through which the exhaled air passes. In the breath sensor 100 in the present example, a part of the light 50 that is emitted by the light emission unit 110 is reflected by the mirror 106, and the first light receiving element 122 receives the reflected light 50. The configuration in the present example makes it possible to reduce a size of the breath sensor 100.

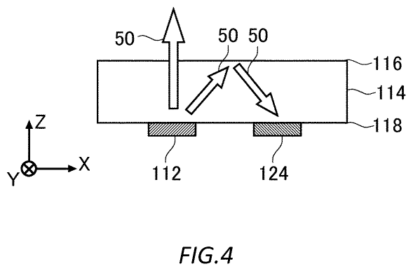

FIG. 4 shows an example of the light emission unit 110. In the present example, the second light receiving element 124 is shown along with the light emission unit 110. The light emission unit 110 may include a light emission element 112 and a substrate 114. The substrate 114 may be transparent with respect to the light 50. The substrate 114 is a GaAs substrate, as an example.

The substrate 114 may have a first main surface 116 and a second main surface 118. The first main surface 116 and the second main surface 118 are two main surfaces which constitute the substrate 114. In the present example, the first main surface 116 is the main surface on a positive side of the Z axis direction, and the second main surface 118 is the main surface on a negative side of the Z axis direction. The first main surface 116 faces the path 20 through which the exhaled air passes.

The light emission element 112 and the second light receiving element 124 may be provided on the same substrate. The light emission element 112 and the second light receiving element 124 in the present example are provided on the second main surface 118 of the substrate 114. The light emission element 112 may have a stacked structure portion of a PN junction or a PIN junction. By supplying power to the stacked structure portion, the light emission element 112 operates as an LED and emits the light 50 of a wavelength corresponding to a bandgap of a material of the stacked structure portion. As an example, the light emission element 112 has, as the stacked structure portion, InAlSb that can output light near the absorption wavelength of carbon dioxide.

A part of the light 50 travels through the substrate 114, passes through the first main surface 116, and is emitted to the path 20. On the other hand, another part of the light 50 travels through the substrate 114, is reflected by the first main surface 116, and is incident on the second light receiving element 124.

The second light receiving element 124 receives the light 50 that has traveled through the substrate 114. The second light receiving element 124 receives the light 50 that has not passed through the path 20 through which the exhaled air passes. This makes it possible to correct an output fluctuation due to the change in the amount of light of the light emission element 112 or due to the temperature.

The second light receiving element 124 may have a stacked structure portion. The stacked structure portion of the second light receiving element 124 may be a diode structure of the PN junction or the PIN junction. The stacked structure portion and a material of the second light receiving element 124 may be similar to those of the stacked structure portion and the material of the light emission element 112. This makes it possible to align the temperature characteristic of the second light receiving element 124 with temperature characteristic of the light emission element 112.

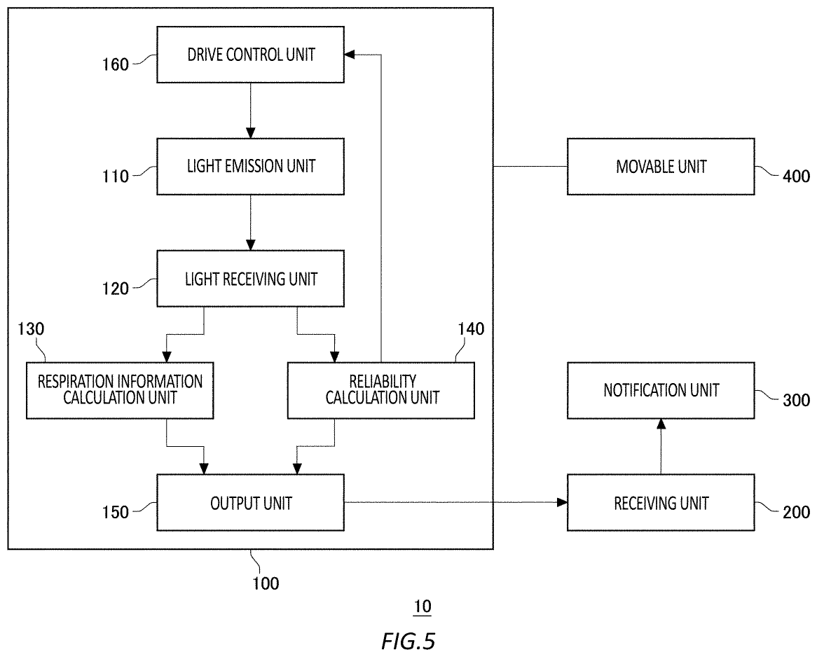

FIG. 5 shows an overview of a configuration of the breath sensing system 10. The breath sensing system 10 in the present example includes the breath sensor 100, a receiving unit 200, and a notification unit 300. The breath sensing system 10 may include a movable unit 400. The breath sensor 100 is the breath sensor 100 described in relation to FIG. 1 to FIG. 4.

For example, the breath sensing system 10 is a system which senses, by the breath sensor 100, the exhalation of a user of the breath sensing system 10, during an execution of application software that operates on a computer inside or outside the breath sensing system 10. The application software may be a game application, or may be a conference application. As an example, by acquiring the respiration information during the execution of the application software, the breath sensing system 10 can analyze a relationship between the psychological information of the user and the respiration information of the user.

The receiving unit 200 receives the respiration information and the reliability level information from the output unit 150 of the breath sensor 100. For example, the receiving unit 200 may receive the respiration information and the reliability level information from the output unit 150, by using short- to medium-range wireless communication technologies such as infrared communication, Bluetooth (registered trademark), or Wi-Fi (registered trademark). The receiving unit 200 may receive the respiration information and the reliability level information from the output unit 150, via a communication network such as LAN (Local Area Network), WAN (Wide Area Network), 5G (5th Generation), 4G (4th Generation), LTE (Long Term Evolution), or WiMax (registered trademark). The receiving unit 200 may receive the respiration information and the reliability level information from the output unit 150, in a wired manner. The receiving unit 200 may supply the received reliability level information to the notification unit 300.

The notification unit 300 provides a notification of the reliability level information. The notification unit 300 may visually provide the notification of the reliability level information. The notification unit 300 may provide the notification of the reliability level information by using at least one of a sound or a vibration. However, a notification method for the reliability level information by the notification unit 300 is not limited to these. The notification unit 300 may provide the notification of the reliability level information by any method. The notification method for the reliability level information by the notification unit 300 will be described below.

The movable unit 400 may have a variable position relative to the path. For example, the user of the breath sensing system 10 moves the movable unit 400, thereby changing the position of the movable unit 400 relative to the path. As another example, the breath sensing system 10 may include a control unit which is able to control the movable unit 400, and the position of the movable unit 400 relative to the path may be changed by the control unit. The breath sensor 100 may be provided in the movable unit 400.

As an example, the movable unit 400 may be a movable unit which connects a sound generation unit of the headset to the breath sensor 100. As another example, the movable unit 400 may be a clip-type apparatus which is able to fasten the breath sensor 100 to clothing or the like, like a lapel microphone. In this case, by changing the position to which the movable unit 400 is fastened, it is possible to change the position of the movable unit 400 relative to the path.

The notification unit 300 may provide the notification of the information related to the movable unit 400, according to the reliability level information. The information related to the movable unit 400 may be an instruction to move the movable unit 400, or may be an instruction not to move the movable unit 400.

The notification unit 300 may provide the notification of the instruction to move the movable unit 400, according to the reliability level information. For example, when the reliability of the exhalation information that is indicated by the reliability level information is lower than a predetermined reference level, the notification unit 300 provides the notification of the instruction to move the movable unit 400. When the reliability of the exhalation information that is indicated by the reliability level information is higher than or equal to the reference level, the notification unit 300 may not provide the notification of the instruction to move the movable unit 400.

For example, the notification of the instruction to move the movable unit 400 is provided, by the notification unit 300, to the user of the breath sensing system 10. The user may move the movable unit 400, according to the notification of the instruction to move the movable unit 400 being provided. The user may move the movable unit 400, until the notification of the instruction to move the movable unit 400 is no longer provided by the notification unit 300. That is, the user may move the movable unit 400, until the reliability of the exhalation information that is indicated by the reliability level information becomes higher than or equal to the reference level. This makes it possible for the user of the breath sensing system 10 to place the breath sensor 100 at a position suitable to sense the exhalation.

The notification unit 300 may provide the notification of the instruction not to move the movable unit 400, according to the reliability level information. Even when the reliability of the exhalation information that is indicated by the reliability level information is lower than a predetermined reference level, it may be possible to enhance the reliability of the exhalation information without moving the movable unit 400. For example, it is possible to enhance the reliability of the exhalation information by a component other than the movable unit 400, by raising light emission intensity of the light emission unit 110 to raise sensitivity of the breath sensor 100, by raising a measurement frequency by the light receiving unit 120, or the like. When it is possible to adjust the reliability of the exhalation information by a component other than the movable unit 400, the notification unit 300 may provide the notification of the instruction not to move the movable unit 400.

As another example, according to the reliability level information, the notification unit 300 may provide the notification of the instruction not to move the movable unit 400 from a position where the reliability of the exhalation information that is indicated by the reliability level information is maximized. When the reliability of the exhalation information at that position is higher than a predetermined reference level, the breath sensing system 10 may not adjust the reliability of the exhalation information. On the other hand, when the reliability of the exhalation information at that position is lower than the predetermined reference level, the breath sensing system 10 may enhance the reliability of the exhalation information by a component other than the movable unit 400, by raising light emission intensity of the light emission unit 110 to raise sensitivity of the breath sensor 100, by raising a measurement frequency by the light receiving unit 120, or the like.

As described above, the breath sensor 100 in the present example includes the reliability calculation unit 140 which calculates the reliability level information and the output unit 150 which outputs the reliability level information; and the breath sensing system 10 in the present example includes the breath sensor 100, the receiving unit 200 which receives the reliability level information, and the notification unit 300 which provides the notification of the reliability level information. This makes it possible for the user of the breath sensing system 10 to place the breath sensor 100 at a position suitable to sense the exhalation. That is, even when the user mounts the breath sensor 100 on an existing system or apparatus to constitute the breath sensing system 10, the user of the breath sensing system 10 can place the breath sensor 100 at a position suitable to sense the exhalation.

It should be noted that the breath sensing system 10 may not include the movable unit 400. In this case, the user of the breath sensing system 10 may provide an instruction to the drive control unit 160 of the breath sensor 100, based on the reliability level information provided by the notification unit 300 in the notification. For example, when the reliability of the exhalation information that is indicated by the reliability level information is low, the user of the breath sensing system 10 may instruct the drive control unit 160 to increase an amount of drive current. In this manner, even when the user mounts the breath sensor 100 on an existing system or apparatus to constitute the breath sensing system 10, the user of the breath sensing system 10 can enhance the reliability of the exhalation information that is acquired by the breath sensor 100.

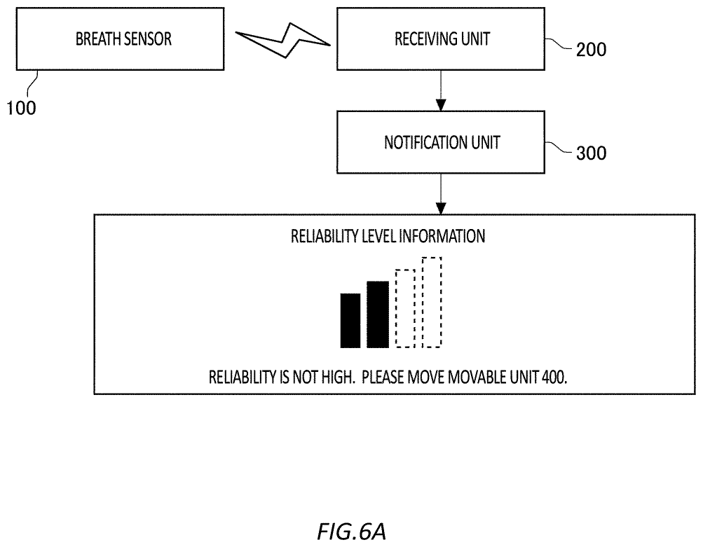

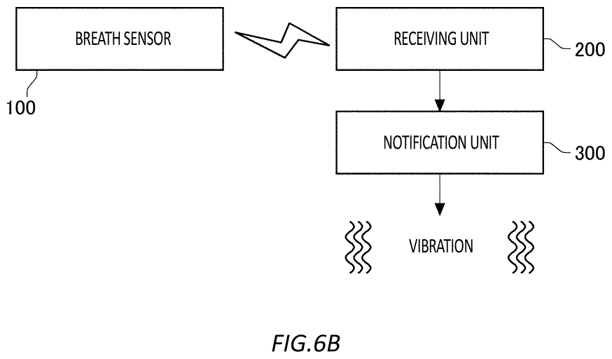

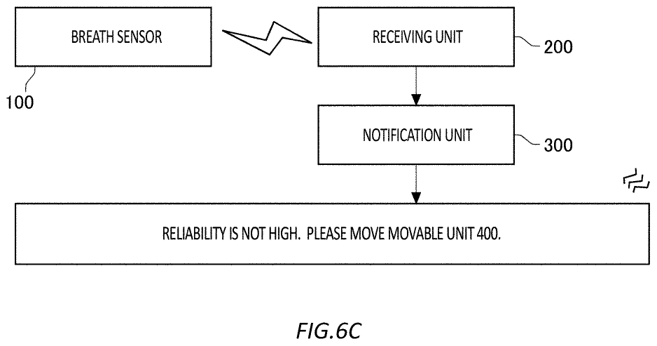

FIG. 6A shows an example of an operation of the breath sensing system 10. The output unit 150 of the breath sensor 100 outputs the respiration information and the reliability level information.

The receiving unit 200 receives the respiration information and the reliability level information from the output unit 150 of the breath sensor 100. The receiving unit 200 may receive the respiration information and the reliability level information, by using a communication technology. The notification unit 300 provides the notification of the reliability level information.

The notification unit 300 in the present example visually provides the notification of the reliability level information. The notification unit 300 may cause a display apparatus to display the reliability level information by text, an illustration, or the like. The notification unit 300 in the present example provides the notification of the reliability level information by the illustration. The notification unit 300 may provide the notification of the reliability level information by changing a display color of a UI. For example, the notification unit 300 may change the display color of the UI to red when the reliability level information is at the first level, may change the display color of the UI to yellow when the reliability level information is at the second level that is higher in reliability than the first level, and may change the display color of the UI to blue when the reliability level information is at the third level that is higher in reliability than the second level.

The display apparatus may be an internal component of the breath sensing system 10, or may be an external component of the breath sensing system 10. That is, the case where the notification unit 300 visually provides the notification of the reliability level information may include a case where the notification unit 300 controls the display apparatus in the breath sensing system 10 to cause the reliability level information to be displayed, and may include a case where the notification unit 300 transmits a display control signal to cause the display apparatus outside the breath sensing system 10 to display the reliability level information.

The reliability calculation unit 140 in the present example calculates the reliability level information in four levels of abstraction. The notification unit 300 may provide, as the reliability level information, the notification indicating which level of the four levels of abstraction the reliability is in. In the present example, the notification unit 300 causes the display apparatus to display the illustration indicating that the reliability is at the second level.

The user of the breath sensing system 10 may move the movable unit 400, according to the reliability level information that is provided by the notification unit 300 in the notification. For example, the user of the breath sensing system 10 moves the movable unit 400, until the reliability level information that is provided by the notification unit 300 in the notification, reaches the third level or a fourth level. This makes it possible for the user of the breath sensing system 10 to enhance the reliability of the exhalation information that is acquired by the breath sensor 100.

The notification unit 300 may visually provide the notification of the instruction to move the movable unit 400. The notification unit 300 may cause a display apparatus to display the instruction to move the movable unit 400 by text, an illustration, or the like. The notification unit 300 in the present example may cause the display apparatus to display the instruction to move the movable unit 400 by text stating that “The reliability is not high. Please move the movable unit 400”. The notification unit 300 may provide the notification of the instruction to move the movable unit 400 by changing a display color of a UI. For example, the notification unit 300 may change the display color of the UI to red when the reliability of the respiration information is low and the movable unit 400 should be moved, and may change the display color of the UI to blue when the reliability of the respiration information is high and the movable unit 400 may not be moved.

The user of the breath sensing system 10 may move the movable unit 400, according to the instruction provided by the notification unit 300 in the notification, to move the movable unit 400. For example, the user of the breath sensing system 10 moves the movable unit 400, until the notification of the instruction to move the movable unit 400 is no longer provided by the notification unit 300. This makes it possible for the user of the breath sensing system 10 to enhance the reliability of the exhalation information that is acquired by the breath sensor 100.

FIG. 6B shows an example of an operation of the breath sensing system 10. The output unit 150 of the breath sensor 100 outputs the respiration information and the reliability level information. The receiving unit 200 receives the respiration information and the reliability level information from the output unit 150 of the breath sensor 100. The receiving unit 200 may receive the respiration information and the reliability level information, by using a communication technology. The notification unit 300 provides the notification of the reliability level information.

The notification unit 300 in the present example provides the notification of the reliability level information by using a vibration. For example, the notification unit 300 may generate the vibration when the reliability of the respiration information that is indicated by the reliability level information is lower than a predetermined reference level, and may not generate the vibration when the reliability of the respiration information that is indicated by the reliability level information is higher than or equal to the reference level. The notification unit 300 may provide the notification of the reliability level information by changing an intensity or a frequency of the generated vibration.

The vibration may be generated by the notification unit 300 itself vibrating, or may be generated by the notification unit 300 causing a vibration apparatus inside or outside the breath sensing system 10 to vibrate. That is, the case where the notification unit 300 provides the notification of the reliability level information by using the vibration may include a case where the notification unit 300 vibrates, may include a case where the notification unit 300 controls and causes the vibration apparatus in the breath sensing system 10 to vibrate, and may include a case where the notification unit 300 transmits a control signal to cause the vibration apparatus outside the breath sensing system 10 to vibrate.

The notification unit 300 may provide the notification of the instruction to move the movable unit 400 by using the vibration. For example, the notification unit 300 may generate the vibration when the reliability of the respiration information is low and the movable unit 400 should be moved, and may not generate the vibration when the reliability is high and the movable unit 400 may not be moved.

The user of the breath sensing system 10 may move the movable unit 400, according to the vibration generated by the notification unit 300. For example, the user of the breath sensing system 10 moves the movable unit 400 until the vibration generated by the notification unit 300 is stopped. This makes it possible for the user of the breath sensing system 10 to enhance the reliability of the exhalation information that is acquired by the breath sensor 100.

FIG. 6C shows an example of an operation of the breath sensing system 10. The output unit 150 of the breath sensor 100 outputs the respiration information and the reliability level information. The receiving unit 200 receives the respiration information and the reliability level information from the output unit 150 of the breath sensor 100. The receiving unit 200 may receive the respiration information and the reliability level information, by using a communication technology. The notification unit 300 provides the notification of the reliability level information.

The notification unit 300 in the present example provides the notification of the reliability level information by using a sound. The notification unit 300 may provide the notification of the reliability level information by using a beep sound, or may provide the notification of the reliability level information by using a voice. For example, the notification unit 300 may generate the beep sound when the reliability of the respiration information that is indicated by the reliability level information is lower than a predetermined reference level, and may not generate the beep sound when the reliability of the respiration information that is indicated by the reliability level information is higher than or equal to the reference level. The notification unit 300 may provide the notification of the reliability level information by changing an intensity or a frequency of the generated beep sound. As another example, the notification unit 300 may provide the notification of the reliability level information by using the voice such as “The reliability of the respiration information is at the second level”.

The sound may be generated by the notification unit 300 itself, or may be generated by the notification unit 300 causing an audio apparatus inside or outside the breath sensing system 10 to generate the sound. That is, the case where the notification unit 300 provides the notification of the reliability level information by using the sound may include a case where the notification unit 300 generates the sound, may include a case where the notification unit 300 controls and causes the audio apparatus in the breath sensing system 10 to generate the sound, and may include a case where the notification unit 300 transmits a control signal to cause the audio apparatus outside the breath sensing system 10 to generate the sound.

The user of the breath sensing system 10 may move the movable unit 400, according to the reliability level information provided by the notification unit 300 in the notification. For example, the user of the breath sensing system 10 moves the movable unit 400 until the beep sound that is generated by the notification unit 300 is stopped, or until the notification unit 300 provides the notification by the voice that the reliability of the respiration information has reached the third level or the fourth level. This makes it possible for the user of the breath sensing system 10 to enhance the reliability of the exhalation information that is acquired by the breath sensor 100.

The notification unit 300 may provide the notification of the instruction to move the movable unit 400 by using the sound. The notification unit 300 may provide the notification of the instruction to move the movable unit 400 by using the beep sound, or may provide the notification of the instruction to move the movable unit 400 by using the voice. The notification unit 300 in the present example provides the notification of the instruction to move the movable unit 400 by using the voice stating that “The reliability is not high. Please move the movable unit 400”. As another example, the notification unit 300 may generate the beep sound when the reliability of the respiration information is low and the movable unit 400 should be moved, and may not generate the beep sound when the reliability of the respiration information is high and the movable unit 400 may not be moved.

The user of the breath sensing system 10 may move the movable unit 400, according to the instruction provided by the notification unit 300 in the notification, to move the movable unit 400. For example, the user of the breath sensing system 10 moves the movable unit 400 until the voice of the instruction to move the movable unit 400 by the notification unit 300 is stopped, or until the beep sound generated by the notification unit 300 is stopped. This makes it possible for the user of the breath sensing system 10 to enhance the reliability of the exhalation information that is acquired by the breath sensor 100.

In the descriptions related to FIG. 6A to FIG. 6C, the descriptions are made such that the notification unit 300 provides the notification visually, or by using the sound or the vibration; however, the notification unit 300 may provide the notification by using another method. When the notification unit 300 provides the notification by using another method, as well, similarly, the notification unit 300 itself may implement that method, or the notification unit 300 may control the internal component of the breath sensing system 10 and cause that method to be implemented, or the notification unit 300 may transmit a control signal to cause the external component of the breath sensing system 10 to implement that method. In addition, the notification unit 300 may provide the notification by any combination of two or more of the visual notification, the notification using the sound, the notification using the vibration, or the notification using another method.

As described above, by mounting the breath sensor 100 on an existing system or apparatus which includes the receiving unit 200 and the notification unit 300, it is possible to easily facilitate the placement of the breath sensor 100 at a position suitable to sense the exhalation. That is, the breath sensor 100 in the present example includes the reliability calculation unit 140 which calculates the reliability level information and the output unit 150 which outputs the reliability level information, and thus when the breath sensor 100 is mounted on an existing system or apparatus which includes the receiving unit 200 and the notification unit 300, by configuring the output unit 150 to communicate with the receiving unit 200, it is possible to easily facilitate the placement of the breath sensor 100 at a position suitable to sense the exhalation.

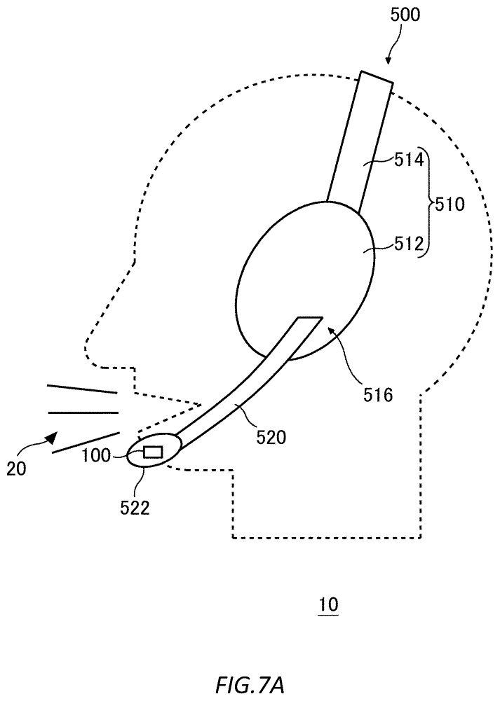

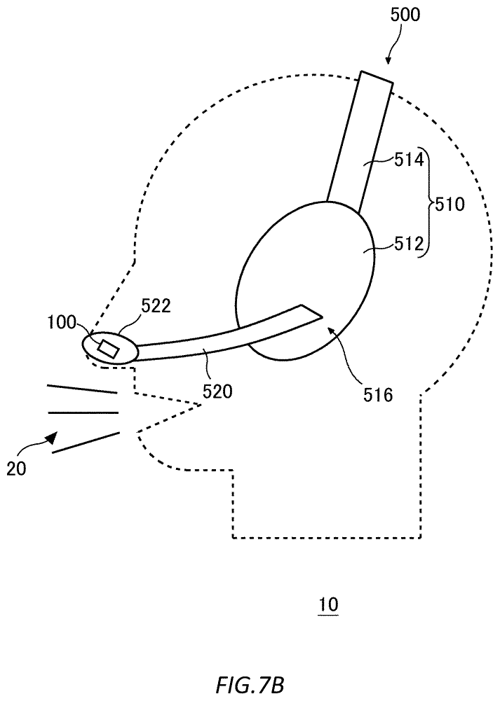

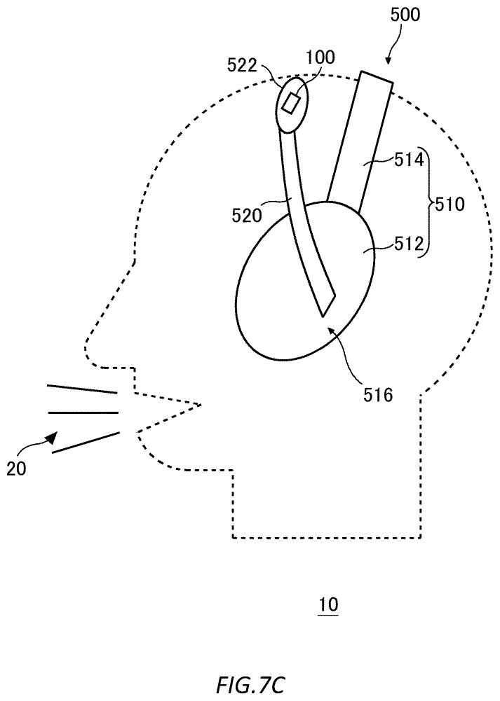

FIG. 7A shows an example of a usage state of a headset 500 including the breath sensor 100. The headset 500 including the breath sensor 100 may constitute a part or all of the breath sensing system 10.

The headset 500 includes a fixed unit 510 and a movable unit 520. As an example, the fixed unit 510 is worn on a head of the user. The fixed unit 510 may include a sound generation unit 512 which generates a sound. The sound generation unit 512 may include a pad portion which contacts an ear of the user when the headset 500 is worn, and a speaker provided in the pad portion. The fixed unit 510 may have two sound generation units 512 corresponding to both ears of the user, and an arm portion 514 which connects two sound generation units 512. The fixed unit 510 may have a connection unit 516 to which the movable unit 520 is connected.

The movable unit 520 is provided to be movable relative to the fixed unit 510. That is, a position of at least a part of the movable unit 520 relative to at least a part of the fixed unit 510 may be changeable. The movable unit 520 in the present example has a rod shape unit which extends from the fixed unit 510. The rod shape unit may be connected to the connection unit 516 of the fixed unit 510. The rod shape unit may be rotatable with respect to the fixed unit 510 with the connection unit 516 as a rotation axis. The movable unit 520 may have a sound sensing unit 522 which is provided at an end portion on an opposite side of the connection unit 516, and which senses a sound. For example, the sound sensing unit 522 is a microphone.

The breath sensor 100 may be provided in the movable unit 520. A position of the breath sensor 100 relative to the fixed unit 510 may be changeable following a movement of the movable unit 520. The breath sensor 100 in the present example is provided near the sound sensing unit 522. By arranging the breath sensor 100 near the sound sensing unit 522, the breath sensor 100 is easily arranged near a mouth of the user, which makes it is possible to enhance the reliability of exhalation information.

The headset 500 may include a communication unit which is able to communicate with the breath sensor 100. The communication unit is an example of the receiving unit 200. The sound generation unit 512 is an example of the notification unit 300. For example, the sound generation unit 512 may provide the notification of the reliability level information, by using the sound. The fixed unit 510 is also an example of the notification unit 300. For example, the fixed unit 510 may provide the notification of the reliability level information, by using the vibration. The movable unit 520 is an example of the movable unit 400. The sound generation unit 512 may provide the notification of the instruction to move the movable unit 520, by using the sound. The fixed unit 510 may provide the notification of the instruction to move the movable unit 520, by using the vibration.

When the headset 500 has the notification unit 300, that is, when the sound generation unit 512 and/or the fixed unit 510 functions as the notification unit 300, the headset 500 including the breath sensor 100 constitutes the entirety of the breath sensing system 10. As another example, the breath sensing system 10 may further include a display apparatus configured to be able to communicate directly or indirectly with the headset 500. In this case, the display apparatus may function as the notification unit 300. However, the display apparatus may be an external component of the breath sensing system 10, and in this case, the notification unit 300 transmits a display control signal for controlling the display apparatus.

As yet another example, the headset 500 may include a display unit which displays the information. The display unit is, for example, a head-mounted display connected to the fixed unit 510. The display unit may be provided to be movable relative to the fixed unit 510. The breath sensor 100 may be provided in the display unit.

The path 20 through which the exhaled air passes may be a path in an open space. The open space may be a space where the exhaled air freely diffuses. For example, when the breath sensor is provided in a case that defines a flow path of the exhalation and the exhaled air is blown into the case, the path through which the exhaled air passes does not correspond to a path in the open space.

The position of the breath sensor 100 may variable in the open space. The breath sensor 100 in the present example is provided in the movable unit 520 of the headset 500, and thus the position is variable in the open space. When the position of the breath sensor 100 is variable in the open space, the reliability of the exhalation information may be decreased, depending on the position of the breath sensor 100. The breath sensor 100 in the present example includes the reliability calculation unit 140 which calculates the reliability level information and the output unit 150 which outputs the reliability level information; and the breath sensing system 10 in the present example includes the breath sensor 100, the receiving unit 200 which receives the reliability level information, and the notification unit 300 which provides the notification of the reliability level information. This makes it possible for the user of the breath sensing system 10 to place the breath sensor 100 at a position suitable to sense the exhalation.

The breath sensor 100 in the present example is arranged near the path 20 in the open space. For example, the reliability calculation unit 140, as the reliability level information, calculates that the reliability of the exhalation information is at the highest level four. The output unit 150 may output the reliability level information, and the receiving unit 200 (for example, the communication unit of the headset 500) may receive the reliability level information. The notification unit 300 (for example, the fixed unit 510 and the sound generation unit 512 of the headset 500, or the display apparatus configured to be able to communicate with the headset 500) may provide the notification of the reliability level information. The reliability of the exhalation information is high, and thus the notification unit 300 may not provide the notification of the instruction to move the movable unit 400 (for example, the movable unit 520 of the headset 500).

FIG. 7B shows an example of a usage state of the headset 500 including the breath sensor 100. The breath sensor 100 in the present example is arranged at a position slightly away from the path 20 in the open space more than in the example in FIG. 7A. For example, the reliability calculation unit 140, as the reliability level information, calculates that the reliability of the exhalation information is at the lower level two that is lower than level four. The output unit 150 may output the reliability level information, and the receiving unit 200 (for example, the communication unit of the headset 500) may receive the reliability level information. The notification unit 300 (for example, the fixed unit 510 and the sound generation unit 512 of the headset 500, or the display apparatus configured to be able to communicate with the headset 500) may provide the notification of the reliability level information. The reliability of the exhalation information is low, and thus the notification unit 300 may provide the notification of the instruction to move the movable unit 400 (for example, the movable unit 520 of the headset 500).

The user of the headset 500 including the breath sensor 100 may move the movable unit 400, according to the reliability level information that is provided by the notification unit 300 in the notification, or the instruction to move the movable unit 400. For example, the user of the headset 500 moves the movable unit 400, until the reliability level information that is provided by the notification unit 300 in the notification, reaches the third level or the fourth level, or until the notification of the instruction to move the movable unit 400 is no longer provided. This makes it possible for the user of the headset 500 to enhance the reliability of the exhalation information that is acquired by the breath sensor 100.

FIG. 7C shows an example of a usage state of the headset 500 including the breath sensor 100. The breath sensor 100 in the present example is arranged at a position away from the path 20 in the open space. The reliability calculation unit 140 may calculate the reliability level information, based on the respiration information. The breath sensor 100 in the present example may not sense the exhalation in the first place, and thus whether or not the reliability of the respiration information is high may be an issue before considering whether or not the signal-to-noise ratio of the light receiving signal is high. Therefore, for example, the reliability calculation unit 140 calculates the reliability level based on the information that the respiration information is not calculated. As an example, the reliability calculation unit 140 calculates that the reliability of the respiration information is at the lowest level one.

The output unit 150 may output the reliability level information, and the receiving unit 200 (for example, the communication unit of the headset 500) may receive the reliability level information. The notification unit 300 (for example, the fixed unit 510 and the sound generation unit 512 of the headset 500, or the display apparatus configured to be able to communicate with the headset 500) may provide the notification of the reliability level information. The reliability of the exhalation information is low, and thus the notification unit 300 may provide the notification of the instruction to move the movable unit 400 (for example, the movable unit 520 of the headset 500).

The user of the headset 500 including the breath sensor 100 may move the movable unit 400, according to the reliability level information that is provided by the notification unit 300 in the notification, or the instruction to move the movable unit 400. For example, the user of the headset 500 moves the movable unit 400, until the reliability level information that is provided by the notification unit 300 in the notification, reaches the third level or the fourth level, or until the notification of the instruction to move the movable unit 400 is no longer provided. This makes it possible for the user of the headset 500 to enhance the reliability of the exhalation information that is acquired by the breath sensor 100.

As described above, by mounting the existing headset 500 with the breath sensor 100, it is possible to easily facilitate the placement of the breath sensor 100 at a position suitable to sense the exhalation. That is, the breath sensor 100 in the present example includes the reliability calculation unit 140 which calculates the reliability level information and the output unit 150 which outputs the reliability level information, and thus when the breath sensor 100 is mounted on the existing headset 500 which includes the receiving unit 200 and the notification unit 300, by configuring the output unit 150 to communicate with the receiving unit 200, it is possible to easily facilitate the placement of the breath sensor 100 at a position suitable to sense the exhalation.

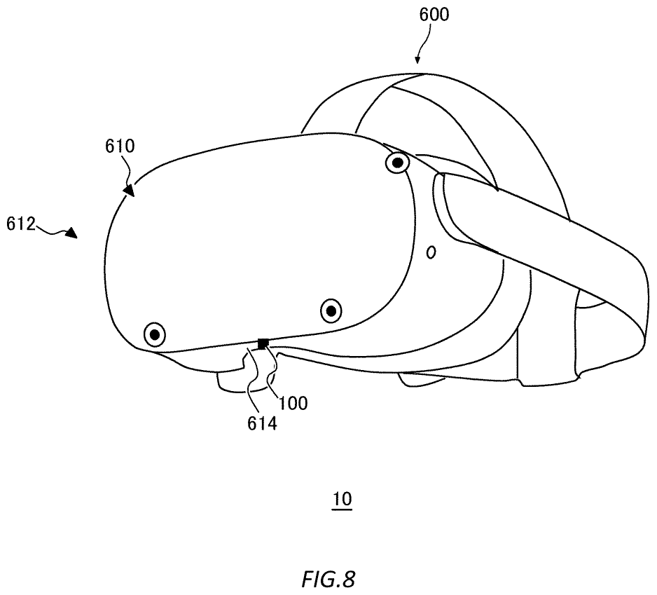

FIG. 8 shows an example of a usage state of a head-mounted display 600 including the breath sensor 100. The head-mounted display 600 including the breath sensor 100 may constitute a part or all of the breath sensing system 10.

The head-mounted display 600 in the present example includes a display unit 610. The breath sensor 100 may be provided in a housing 612 of the display unit 610. The breath sensor 100 may be provided on a surface of the housing 612. The breath sensor 100 in the present example is provided on a bottom surface 614 of the housing 612. When the head-mounted display 600 includes a voice acquisition unit which acquires a voice of the user, the breath sensor 100 may be arranged in the voice acquisition unit, or may be arranged near the voice acquisition unit.

The head-mounted display 600 may include a communication unit which is able to communicate with the breath sensor 100. The communication unit is an example of the receiving unit 200. The display unit 610 is an example of the notification unit 300. For example, the display unit 610 may visually provide the notification of the reliability level information. The housing 612 is also an example of the notification unit 300. For example, the housing 612 may provide the notification of the reliability level information, by using the vibration. In the present example, as well, the path 20 through which the exhaled air passes may be a path in an open space.

The head-mounted display 600 in the present example does not include a movable unit. That is. the breath sensing system 10 may not include the movable unit 400. In this case, the drive control unit 160 may control the drive of the light emission unit 110 based on the reliability level information. Alternatively, the user of the head-mounted display 600 may provide the instruction to the drive control unit 160 of the breath sensor 100, based on the reliability level information provided by the notification unit 300 in the notification. For example, when the reliability of the exhalation information that is indicated by the reliability level information is low, the user of the head-mounted display 600 may instruct the drive control unit 160 to increase an amount of drive current. In this manner, even when the user mounts the breath sensor 100 on the head-mounted display 600 to constitute the breath sensing system 10, the user of the breath sensing system 10 can enhance the reliability of the exhalation information that is acquired by the breath sensor 100. However, the head-mounted display 600 may have a movable unit, and the breath sensor 100 may be provided in the movable unit.

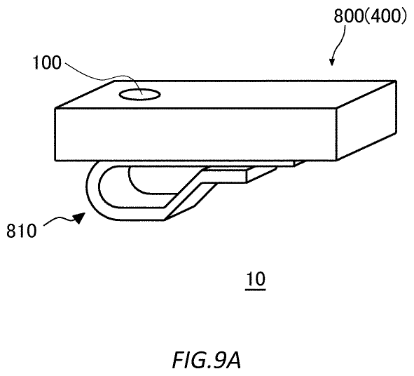

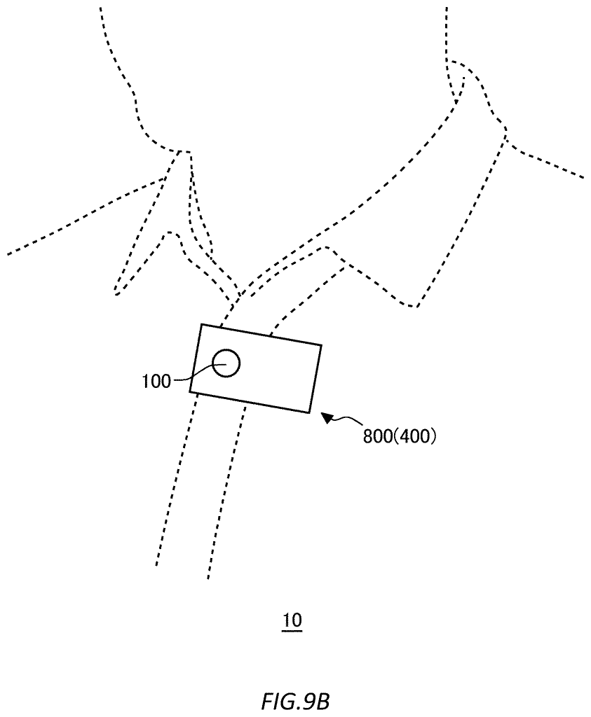

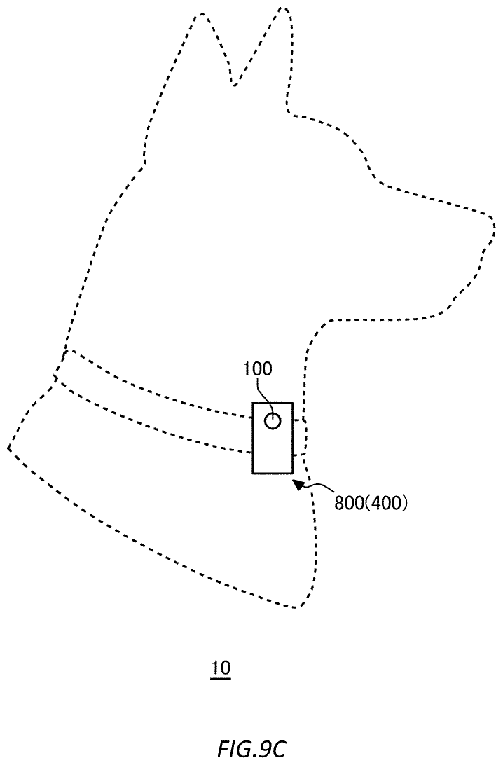

FIG. 9A shows an example of a clip-type apparatus 800 including the breath sensor 100. The clip-type apparatus 800 including the breath sensor 100 may constitute a part or all of the breath sensing system 10.

The clip-type apparatus 800 includes a fastener 810. The clip-type apparatus 800 may be attached to clothing or the like by the fastener 810. The clip-type apparatus 800 is an example of the movable unit 400. That is, by changing a position to which the movable unit 400 (clip-type apparatus 800) is fastened, it is possible to change the position of the movable unit 400 relative to the path.

FIG. 9B shows an example of a usage state of the clip-type apparatus 800 including the breath sensor 100. The clip-type apparatus 800 may be attached to clothing. In this manner, the breath sensor 100 may sense the exhaled air generated by the respiration of a human.

FIG. 9C shows an example of a usage state of the clip-type apparatus 800 including the breath sensor 100. The clip-type apparatus 800 may be attached to a collar of a pet or the like including a dog or a cat. In this manner, the breath sensor 100 may sense exhaled air generated by respiration of a living body, such as a pet including the dog or the cat.

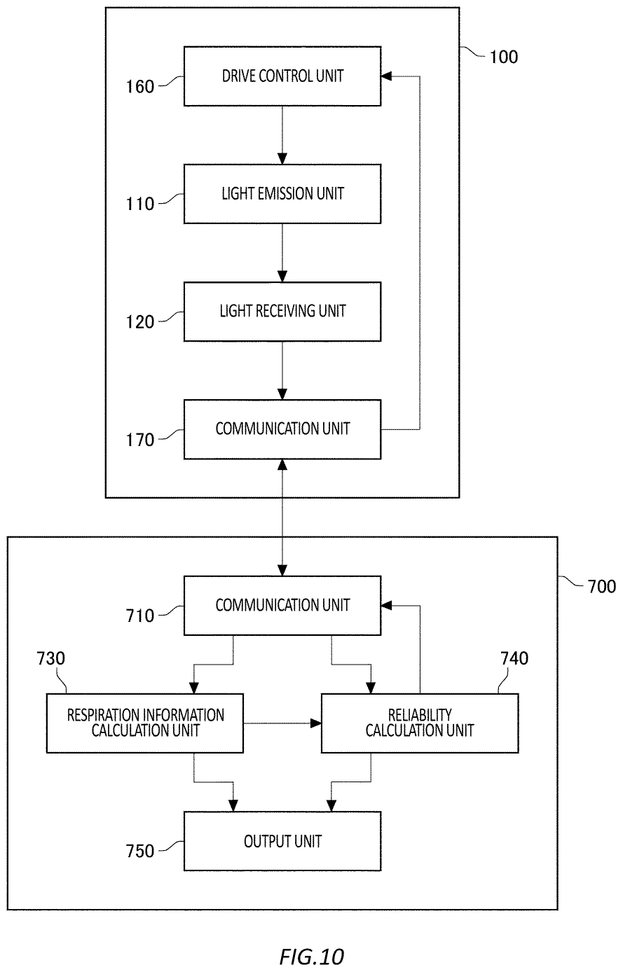

FIG. 10 shows an overview of a configuration of the breath sensor 100 and a calculation apparatus 700. The breath sensor 100 in the present example includes the light emission unit 110 and the light receiving unit 120. The breath sensor 100 may include the drive control unit 160 and a communication unit 170. The calculation apparatus 700 includes a respiration information calculation unit 730, a reliability calculation unit 740, and an output unit 750. The calculation apparatus 700 may include a communication unit 710.

The light emission unit 110, the light receiving unit 120, and the drive control unit 160 may be the same as the light emission unit 110, the light receiving unit 120, and the drive control unit 160 described in relation to FIG. 1 to FIG. 8. The respiration information calculation unit 730, the reliability calculation unit 740, and the output unit 750 may be the same as the respiration information calculation unit 130, the reliability calculation unit 140, and the output unit 150 described in relation to FIG. 1 to FIG. 8. That is, the respiration information calculation unit 130, the reliability calculation unit 140, and the output unit 150 described in relation to FIG. 1 to FIG. 8 may not necessarily be provided as internal components of the breath sensor 100, and may be provided as components of the external calculation apparatus 700.

The communication unit 170 may transmit the light receiving signal output by the light receiving unit 120 to the calculation apparatus 700. For example, the communication unit 170 transmits the light receiving signal to the communication unit 710 of the calculation apparatus 700. The communication between the communication unit 170 and the communication unit 710 may be communication using short- to medium-range wireless communication technologies such as infrared communication, Bluetooth (registered trademark), or Wi-Fi (registered trademark). The communication between the communication unit 170 and the communication unit 710 may be communication via a communication network such as a LAN, a WAN, a 5G, a 4G, an LTE, or a WiMax (registered trademark). The communication between the communication unit 170 and the communication unit 710 may also be wired communication. The communication unit 170 is an example of a transmission unit which transmits the information in a wired manner. The communication unit 170 is an example of the transmission unit which transmits the information wirelessly. It should be noted that the light receiving unit 120 may directly output the light receiving signal to the calculation apparatus 700. In this case, the light receiving unit 120 is an example of the transmission unit.

The communication unit 710 may receive the light receiving signal. The communication unit 710 may supply the received light receiving signal to the respiration information calculation unit 730 and the reliability calculation unit 740. It should be noted that the respiration information calculation unit 730 and the reliability calculation unit 740 may be directly supplied with the light receiving signals from the light receiving unit 120, by the communication via a short-range wireless communication technology, a communication network, or a wired communication technology.

The reliability calculation unit 740 may supply the calculated reliability level information to the communication unit 710. The communication unit 710 may transmit the reliability level information to the communication unit 170. The communication unit 170 may receive the reliability level information. The communication unit 170 may supply the received reliability level information to the drive control unit 160. The drive control unit 160 may control the drive of the light emission unit 110 based on the reliability level information.

As described above, at least some of the components of the breath sensor 100 described in relation to FIG. 1 to FIG. 8 may not necessarily be provided as an internal component of the breath sensor 100. The breath sensor 100 may be implemented as a complex apparatus obtained by appropriately combining a sensor including a light emission element and a light receiving element, a control apparatus which controls a drive of the sensor, and a processing apparatus which processes a signal of the sensor.

The breath sensor 100 may be provided in the movable unit 400. The calculation apparatus 700 may include the receiving unit 200 and the notification unit 300. That is, the breath sensing system 10 may be constituted by the breath sensor 100 provided in the movable unit 400 and the calculation apparatus 700.

Configurations other than that in which the breath sensor 100 having the light emission unit 110 and the light receiving unit 120 is provided in the movable unit 400, are optional. The respiration information calculation unit 130 may be provided in the breath sensor 100, may be provided in the calculation apparatus 700, or may be provided in both of the breath sensor 100 and the calculation apparatus 700. The same applies to the reliability calculation unit 140, the output unit 150, and the notification unit 300.

Various embodiments of the present invention may be described with reference to flowcharts and block diagrams whose blocks may represent (1) steps of processes in which operations are performed or (2) sections of apparatuses responsible for performing operations. Certain stages and sections may be implemented by a dedicated circuit, a programmable circuit supplied together with computer-readable instructions stored on computer-readable media, and/or processors supplied together with computer-readable instructions stored on computer-readable media. The dedicated circuit may include digital and/or analog hardware circuits, and may include integrated circuits (IC) and/or discrete circuits. The programmable circuit may include a reconfigurable hardware circuit including logical AND, logical OR, logical XOR, logical NAND, logical NOR, and other logical operations, a memory element or the like such as a flip-flop, a register, a field programmable gate array (FPGA) and a programmable logic array (PLA), or the like.

A computer-readable medium may include any tangible device that can store instructions to be executed by a suitable device, and as a result, the computer-readable medium having instructions stored thereon includes a product including instructions that can be executed in order to create means for executing operations designated in the flowcharts or block diagrams. Examples of the computer-readable medium may include an electronic storage medium, a magnetic storage medium, an optical storage medium, an electromagnetic storage medium, a semiconductor storage medium, and the like. More specific examples of the computer-readable medium may include a floppy (registered trademark) disk, a diskette, a hard disk, a random access memory (RAM), a read-only memory (ROM), an erasable programmable read-only memory (EPROM or flash memory), an electrically erasable programmable read-only memory (EEPROM), a static random access memory (SRAM), a compact disc read-only memory (CD-ROM), a digital versatile disc (DVD), a Blu-ray (registered trademark) disc, a memory stick, an integrated circuit card, or the like.

The computer-readable instruction may include: an assembler instruction, an instruction-set-architecture (ISA) instruction; a machine instruction; a machine dependent instruction; a microcode; a firmware instruction; state-setting data; or either a source code or an object code written in any combination of one or more programming languages, including an object oriented programming language such as Smalltalk (registered trademark), JAVA (registered trademark), C++, or the like, and a conventional procedural programming language such as a “C” programming language or a similar programming language.

The computer-readable instruction may be provided for a processor or programmable circuit of a programmable data processing apparatus, such as a computer, locally or via a local area network (LAN), a wide area network (WAN) such as the Internet, or the like to execute the computer-readable instruction in order to create means for executing the operations specified in the flowcharts or block diagrams. Here, the computer may be a personal computer, or PC (personal computer), a tablet computer, a smartphone, a workstation, a server computer, a general purpose computer, a special purpose computer, or the like, or may be a computer system to which a plurality of computers are connected. Such computer system to which the plurality of computers are connected is also referred to as a distributed computing system, and is a computer in a broad sense. In the distributed computing system, a plurality of computers collectively execute a program by each of the plurality of computers executing a portion of the program, and passing data during the execution of the program among the computers as needed.

Examples of the processor include a computer processor, a central processing unit (CPU), a processing unit, a microprocessor, a digital signal processor, a controller, a microcontroller, and the like. The computer may include one processor or a plurality of processors. In a multiprocessor system including a plurality of processors, the plurality of processors collectively execute a program by each of the processors executing a portion of the program, and passing data during the execution of the program among the processors as needed. For example, in execution of multiple tasks, each of the plurality of processors may execute a portion of each task pieces by pieces by performing task-switching for each time slice. In this case, which portion of one program each processor is responsible for executing dynamically changes. Moreover, which portion of the program each of the plurality of processor is responsible for executing may be determined statically by multiprocessor-aware programming.

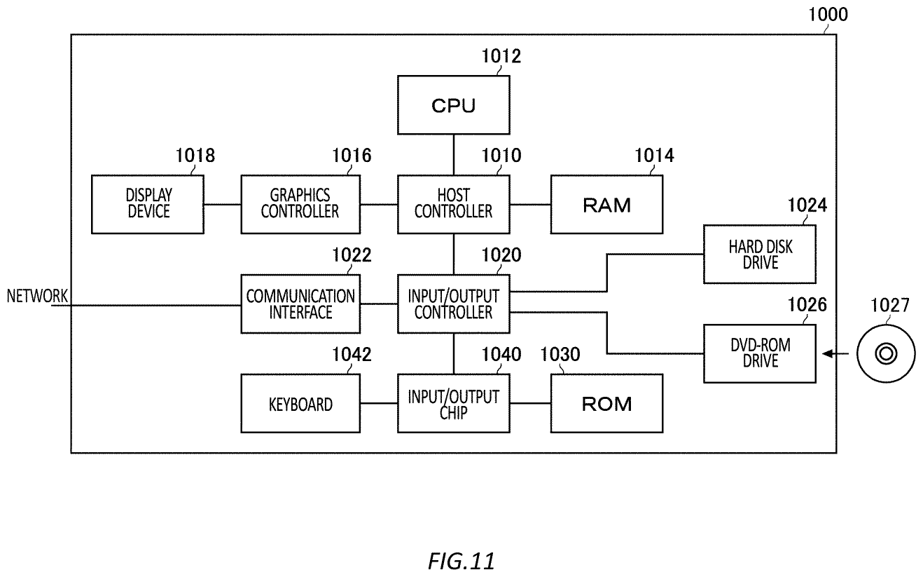

FIG. 11 shows an example of a computer 1000 in which a plurality of aspects of the present invention may be embodied entirely or partially. A program installed in the computer 1000 can cause the computer 1000 to function as an operation associated with the apparatuses according to the embodiments of the present invention or as one or more sections of the apparatuses, or can cause the operation or the one or more sections to be executed, and/or can cause the computer 1000 to execute a process according to the embodiments of the present invention or a step of the process. Such programs may be executed by a CPU 1012 to cause the computer 1000 to perform specific operations associated with some or all of the blocks in the flowcharts and block diagrams described in the present specification.

The computer 1000 according to the present embodiment includes the CPU 1012, a RAM 1014, a graphics controller 1016, and a display device 1018, which are interconnected by a host controller 1010. The computer 1000 also includes input/output units such as a communication interface 1022, a hard disk drive 1024, a DVD-ROM drive 1026, and an IC card drive, which are connected to the host controller 1010 via an input/output controller 1020. The computer also includes legacy input/output units such as a ROM 1030 and a keyboard 1042, which are connected to the input/output controller 1020 via an input/output chip 1040.

The CPU 1012 operates according to programs stored in the ROM 1030 and the RAM 1014, thereby controlling each unit. The graphics controller 1016 acquires image data generated by the CPU 1012 in a frame buffer or the like provided in the RAM 1014 or in itself, and causes the image data to be displayed on the display device 1018.

The communication interface 1022 communicates with another electronic device via a network. The hard disk drive 1024 stores programs and data used by the CPU 1012 in the computer 1000. The DVD-ROM drive 1026 reads the programs or the data from a DVD-ROM 1027, and provides the programs or the data to the hard disk drive 1024 via the RAM 1014. The IC card drive reads the programs and the data from an IC card, and/or writes the programs and the data to the IC card.

The ROM 1030 stores therein boot programs and the like executed by the computer 1000 at the time of activation, and/or programs that depend on the hardware of the computer 1000. The input/output chip 1040 may also connect various input/output units to the input/output controller 1020 via a parallel port, a serial port, a keyboard port, a mouse port, or the like.

Programs are provided by a computer-readable medium such as the DVD-ROM 1027 or the IC card. The programs are read from the computer-readable medium, are installed in the hard disk drive 1024, the RAM 1014, or the ROM 1030 which is also an example of the computer-readable medium, and are executed by the CPU 1012. Information processing written in these programs is read by the computer 1000, and provides cooperation between the programs and the various types of hardware resources described above. The apparatus or method may be constituted by implementing operations or processing of information according to the usage of the computer 1000.

For example, in a case where communication is performed between the computer 1000 and an external device, the CPU 1012 may execute a communication program loaded in the RAM 1014 and instruct the communication interface 1022 to perform communication processing based on processing written in the communication program. Under the control of the CPU 1012, the communication interface 1022 reads transmission data stored in a transmission buffer processing area provided in a recording medium such as the RAM 1014, the hard disk drive 1024, the DVD-ROM 1027, or the IC card, transmits the read transmission data to the network, or writes reception data received from the network in a reception buffer processing area or the like provided on the recording medium.

In addition, the CPU 1012 may cause the RAM 1014 to read all or a necessary part of a file or database stored in an external recording medium such as the hard disk drive 1024, the DVD-ROM drive 1026 (DVD-ROM 1027), the IC card, or the like, and may execute various types of processing on data on the RAM 1014. Then, the CPU 1012 writes the processed data back in the external recording medium.

Various types of information such as various types of programs, data, tables, and databases may be stored in a recording medium and subjected to information processing. The CPU 1012 may execute, on the data read from the RAM 1014, various types of processing including various types of operations, information processing, conditional judgement, conditional branching, unconditional branching, information retrieval/replacement, or the like described throughout the present disclosure and specified by instruction sequences of the programs, and writes the results back to the RAM 1014. In addition, the CPU 1012 may search for information in a file, a database, or the like in the recording medium. For example, when a plurality of entries, each having an attribute value of a first attribute associated with an attribute value of a second attribute, are stored in the recording medium, the CPU 1012 may retrieve, out of the plurality of entries, an entry with the attribute value of the first attribute specified that meets a condition, read the attribute value of the second attribute stored in that entry, and thereby acquiring the attribute value of the second attribute associated with the first attribute meeting a predetermined condition.

The program or software module described above may be stored in the computer-readable medium on the computer 1000 or near the computer 1000. In addition, a recording medium such as a hard disk or a RAM provided in a server system connected to a dedicated communication network or the Internet can be used as a computer-readable medium, thereby providing a program to the computer 1000 via the network.

While the present invention has been described by way of the embodiments, the technical scope of the present invention is not limited to the above-described embodiments. It is apparent to persons skilled in the art that various alterations or improvements can be made to the above described embodiments. It is also apparent from description of the claims that the embodiments to which such modifications or improvements are made may be included in the technical scope of the present invention.

It should be noted that each process of the operations, procedures, steps, stages, and the like performed by the apparatus, system, program, and method shown in the claims, specification, or drawings can be executed in any order as long as the order is not indicated by “prior to”, “before”, or the like and as long as the output from a previous process is not used in a later process. Even if the operation flow is described using phrases such as “first” or “next” for the sake of convenience in the claims, specification, or drawings, it does not necessarily mean that the process must be performed in this order.

### EXPLANATION OF REFERENCES

10: breath sensing system; 20: path; 50: light; 100: breath sensor; 102: support member; 104: calculation unit; 106: mirror; 110: light emission unit; 112: light emission element; 114: substrate; 116: first main surface; 118: second main surface; 120: light receiving unit; 122: first light receiving element; 124: second light receiving element; 126: optical filter; 130: respiration information calculation unit; 140: reliability calculation unit; 150: output unit; 160: drive control unit; 170: communication unit; 200: receiving unit; 300: notification unit; 400: movable unit; 500: headset; 510: fixed unit; 512: sound generation unit; 514: arm portion; 516: connection unit; 520: movable unit; 522: sound sensing unit; 600: head-mounted display; 610: display unit; 612: housing; 614: bottom surface; 700: calculation apparatus; 710: communication unit; 730: respiration information calculation unit; 740: reliability calculation unit; 750: output unit; 800: clip-type apparatus; 810: fastener; 1000: computer; 1010: host controller; 1012: CPU; 1014: RAM; 1016: graphics controller; 1018: display device; 1020: input/output controller; 1022: communication interface; 1024: hard disk drive; 1026: DVD-ROM drive; 1027: DVD-ROM; 1030: ROM; 1040: input/output chip; 1042: keyboard.

What is claimed is:

1. A breath sensing system comprising:
a breath sensor which senses exhaled air generated by respiration, the breath sensor having
a light emission unit which emits light that is directed toward a path through which the exhaled air passes, and
a light receiving unit which receives at least a part of the light emitted by the light emission unit, and which outputs a light receiving signal corresponding to a light receiving result;
a respiration information calculation unit which calculates respiration information related to the respiration, based on the light receiving signal;
a reliability calculation unit which calculates reliability level information indicating reliability of the respiration information, based on a signal or a signal-to-noise ratio of the light receiving signal;
an output unit which outputs the respiration information and the reliability level information;
a receiving unit which receives the respiration information and the reliability level information from the output unit;
a movable unit which is provided with the breath sensor, and which has a variable position relative to the path; and
a notification unit which provides a notification of information related to the movable unit, according to the reliability level information received by the receiving unit.

2. The breath sensing system according to claim 1, wherein
the reliability calculation unit calculates the reliability level information based on the signal-to-noise ratio of the light receiving signal.

3. The breath sensing system according to claim 1, wherein
the reliability calculation unit calculates the reliability level information based on the respiration information.

4. The breath sensing system according to claim 1, wherein
the reliability calculation unit, as the reliability level information,
calculates, when the reliability of the respiration information falls within a predetermined first range, that the reliability is at a first level, and
calculates, when the reliability of the respiration information falls within a second range that has reliability higher than the first range, that the reliability is at a second level.

5. The breath sensing system according to claim 1, comprising
a drive control unit which controls a drive of the light emission unit based on the reliability level information.

6. The breath sensing system according to claim 5, wherein
the reliability level information includes the signal-to-noise ratio of the light receiving signal, and
the drive control unit controls an amount of drive current for driving the light emission unit based on the signal-to-noise ratio.

7. The breath sensing system according to claim 6, wherein
the drive control unit causes the amount of the drive current to be increased when the signal-to-noise ratio falls below a predetermined reference value.

8. The breath sensing system according to claim 1, wherein
the respiration information calculation unit calculates at least one of a period or a duration of the respiration.

9. The breath sensing system according to claim 1, wherein
the output unit outputs the reliability level information at a time interval that is longer than a time interval at which the respiration information is output.

10. The breath sensing system according to claim 1, wherein
the breath sensor has a transmission unit which transmits information in a wired manner.

11. The breath sensing system according to claim 2, wherein
the breath sensor has a transmission unit which transmits information in a wired manner.

12. The breath sensing system according to claim 1, wherein
the breath sensor has a transmission unit which transmits information wirelessly.

13. The breath sensing system according to claim 2, wherein
the breath sensor has a transmission unit which transmits information wirelessly.

14. The breath sensing system according to claim 1, wherein
the notification unit provides a notification of an instruction to move the movable unit, according to the reliability level information.

15. The breath sensing system according to claim 1, wherein
the notification unit visually provides a notification of the reliability level information.

16. The breath sensing system according to claim 1, wherein
the notification unit provides a notification of the reliability level information by using at least one of a sound or a vibration.

17. The breath sensing system according to claim 1, wherein
the path through which the exhaled air passes is a path in an open space.

18. The breath sensing system according to claim 17, wherein
a position of the breath sensor is variable in the open space.

19. A breath sensing method for sensing exhaled air generated by respiration, the breath sensing method comprising:
emitting light that is directed toward a path through which the exhaled air passes;
receiving at least a part of the light that is emitted, and outputting a light receiving signal corresponding to a light receiving result;
calculating respiration information related to the respiration, based on the light receiving signal;
calculating reliability level information indicating reliability of the respiration information, based on the light receiving signal; and
outputting the respiration information and the reliability level information.

20. A non-transitory computer-readable medium having recorded thereon a program that, when executed by a computer, causes the computer to:
control a light emission unit to emit light that is directed toward a path through which exhaled air passes;
control a light receiving unit to receive at least a part of the light emitted by the light emission unit, and to output a light receiving signal corresponding to a light receiving result;
calculate respiration information related to respiration, based on the light receiving signal;
calculate reliability level information indicating reliability of the respiration information, based on the light receiving signal; and
output the respiration information and the reliability level information.
